SEQUENCE LISTING (2 sequences)

SEQ ID NO: 1 - DNA, 20 bp, Artificial Sequence
  primer_bind: synthetic oligonucleotide primer complementary to viral sequence
cgagtcaacacgggtgaaga

SEQ ID NO: 2 - DNA, 20 bp, Artificial Sequence
  primer_bind: synthetic oligonucleotide primer complementary to viral sequence
cgtgtcagtcatgtgtcgct

(19) United States
(12) Patent Application Publication
(10) Pub. No.: US 2002/0159993 A1
(43) Pub. Date: Oct. 31, 2002

(54) Immortal cell line derived from grouper epinephelus coioides and its applications therein

(76) Inventor: Shau-Chi Chi, Taipei (TW)

Correspondence Address: VENABLE, BAETJER, HOWARD AND CIVILETTI, LLP, P.O. BOX 34385, WASHINGTON, DC, 20043-9998, US

(21) Appl. No.: 10/004,414

(22) Filed: Dec. 6, 2001

Related U.S. Application Data
(63) Continuation-in-part of application No. 09/450,696, filed on Nov. 30, 1999.
(60) Provisional application No. 60/110,699, filed on Dec. 3, 1998.

(51) Int. Cl. A61K 39/395; A61K 39/42; C12N 5/00; C12N 5/02; C12N 5/06; C12N 5/16

(52) U.S. Cl. 424/130.1; 435/325; 435/345; 435/339; 424/147.1; 424/141.1; 424/159.1

(57) ABSTRACT

The present invention provides for antibodies against nerous necrosis virus (NNV) and infectious pancreatic necrosis (IPNV) virus. The antibodies include polyclonal and monoclonal antibodies. NNV and IPNV are produced from an immortal cell line derived from Epinephelus coioides having an ATCC deposit number of PTA-859. The present invention also provides methods for detecting viral infections in fish using enzyme immunoassay (EIA).

### RELATED APPLICATION

This application is a continuation-in-part application which claims the priority of U.S. Provisional Patent Application No. 60/110,699, filed on Dec. 3, 1998, and U.S. patent application Ser. No. 09/450,696, filed on Nov. 30, 1999, which are incorporated herein by reference.

### FIELD OF THE INVENTION

The present invention relates to antibodies against Infectious Pancreatic Necrosis Virus (IPNV) and Nervous Necrosis Virus (NNV) and methods for preparing the antibodies. The antibodies include monoclonal and polyclonal antibodies. Both the IPNV and NNV are mass-produced in an immortal cell line (GF-1) derived from the fin tissue of grouper Epinephelus coioides. The present invention also relates to the method for detecting NNV or IPNV using immunoassay, particularly enzyme immunoassay method (EIA).

### BACKGROUND OF THE INVENTION

Nervous necrosis virus (NNV), a pathogen found in many varieties of hatchery-reared marine fish, has caused mass mortality of such fish at their larval or juvenile stages. NNV belongs to the family Nodaviridae. Fish nodaviruses isolated from different species (such as SJNNV, BFNNV, JFNNV, TPNNV, RGNNV, GNNV etc.) are closely related to each other owing to the high similarity of the conserved region of their coat protein genes. NNV, also named as fish encephalitis virus (FEV) and piscine neuropathy nodavirus (PNN), is an unenveloped spherical virus with particles sized between 25 and 34 nm. The virus is characterized by vacuolation of the nerve tissues. Viral Nervous Necrosis (VNN) disease has been found in many countries under various names such as viral fish encephalitis, fish encephalomyelitis, cardiac myopathy syndrome. The hosts of NNV include many species of marine fish, for example: parrotfish, sea bass, turbot, grouper, stripped jack, tiger puffer, berfin flounder, halibut, barramundi, and spotted wolffish.

According to the statistics shown in 1993, approximately 159 fish cell lines have been established which have demonstrated a capacity for growing fish viruses (Fryer and Lannan, J. Tissue Culture Method (1994), 10:57-94). Most of these cell lines are derived from the tissues of freshwater fish. There are only thirty-four cell lines that are originated from marine fish. Although some of the fish cell lines, which include RTG-2, CHSE-214, BF2, SBL, FHM, EPC, have been tested for the susceptibility of fish nodavirus, none of these cells lines has shown cytopathic effects (CPE) after viral inoculations.

In 1996, SSN-1 cell line, a cell line derived from striped snakehead Channa Striatus, has been successfully used for isolating sea bass nodavirus (Frerichs et al., J. General Virology (1996)77:20672071). However, SSN-1 cell line has been known to be persistently contaminated with C-type retrovirus (Frerichs et al., J. General Virology (1991) 72:2537-2539). Therefore, it is not suitable for the production of fish nodavirus.

Infectious pancreatic necrosis virus (IPNV) is the prototype virus of the family Bimaviridae. Bimaviruses also include infectious bursal disease virus of domestic fowl and drosophila X virus of Drosophila melanogaster. The virus is capable of infecting a number of different hosts and has a worldwide presence. For example, IPNV has been found in a variety of fish species throughout the world, including various trout and salmon species, carp, perch, pike, eels and char, as well as mollusks and crustaceans, although so far acute diseases has only been reported in a limited number of salmonid species, such as trout and salmon. Pilcher et al., Crit. Rev. Microbiol. (1980) 7:287-364.

An outbreak of IPNV in a hatchery can be an economic disaster for the aquaculturist. IPNV attacks young fish (usually two- to four-month old), resulting in high mortality. Frantsi et al., J. Wildlife Dis. (1971)&:249-255. In trout, IPNV usually attacks young fry about five to six weeks after their first feeding. The affected fish are darker than usual, have slightly bulging eyes and often have swollen bellies. At the beginning of an outbreak, large numbers of slow, dark fry are seen up against water outflows, and fish are seen “shivering” near the surface. The pancreas appears to be the primary target organ for the virus, with the pancreatic fat cells or Islets of Langerhans being unaffected. The intestine is the only organ besides the pancreas where viral lesions are consistently found. McKnight et al., Br. Vet. J. (1976)132:76-86.

After an IPNV outbreak, the surviving fish generally become carriers of the virus. The persistence of the virus in carrier fish appears to be due to continuous viral production by a small number of infected cells in certain organs. The surviving carrier fish become even more economically disastrous to aquaculturists because the only control method currently available for eliminating the virus in carrier fish is to completely destroy these fish.

Viral diseases cannot be cured by therapeutic reagents. The best ways to contain viral diseases include prevention through early detection and the development of vaccines. In either way, the understanding of the biological, biochemical, and serological characteristics of the virus is fundamentally required, which in turn relies on the industry to have the capacity of mass producing the pure form of viruses, preferably through an in vitro cell culture system. Therefore, the development of a new cell line which can be susceptible to fish viruses, particularly NNV and IPNV, is desperately in demand in order to control the wide spread of fish viral diseases due to viral infection.

Grouper is an important hatchery fish in Taiwan. In recent years, there have been several reports regarding the establishment of cell lines derived from grouper. For example, Chen et. al. (Japan Scientific Society Press (Tokyo) (1988) 218-227) have reported their establishment of several cell lines from the fin and kidney tissues of grouper Epinephelus awoara. Lee (Master Thesis from the Department of Zoology at the National Taiwan University, 1993) also has reported his establishment of the cell lines derived from the eye pigment cells and brain tissue of grouper Epinephelus amblycephalus. However, Chen et al. do not provide sufficient data in support of the claim for immortality in their cell lines and Lee expressly indicates in his thesis that his grouper cell lines are not immortal. Moreover, neither Chen et al.'s nor Lee's cell lines are susceptible to fish nodavirus.

Recently, severe mortality among groupers has repeatedly occurred which is caused primarily by nodavirus. As present, fish nodavirus has been discovered in grouper and can be isolated from moribund grouper which possess symptoms of VNN disease (Chi et al., J. Fish Disease (1997) 20:185-193). Electron microscopic examination of the tissues from grouper shows that, in addition to nodavirus infection, grouper is susceptible to other viral infections (Chi, COA Fisheries Series No.61, Reports on Fish Disease Research (1997) 18:59-69).

IPNV is not a virus commonly found groupers. IPNV was originally reported from Canada as catarrhal enteritis in brook trout (Salvelinus fontinalis) and later from USA as an acute viral disease in hatchery-reared brook trout fry at start feeding. Since then, the disease has spread to most salmonid farming countries. The first report of IPNV-related pathology in Atlantic salmon came from Scotland. The same year clinical IPNV with high mortality in post-smolt and IPNV with 30% mortality in an Atlantic salmon hatchery were reported from Norway. In Norway, the incidence of clinical infectious pancreatic necrosis (IPN) disease in Atlantic salmon farms was 39% in 1991, and it had increased to 61% in 1995. Christie, Fish Vaccinology (1997) 90:191-199.

At this time, there are a few cell lines that are capable of producing IPNV. The most commonly known one is Chinook salmon embryo (CHSE) cell line, which is available commercially and can be obtained from the American Type Culture Collection (USA). Recently, there has been reported that another cell line derived from Penaeus Monodon is also susceptible to IPNV. See U.S. Pat. No. 6,143,547 to Hsu. However, although these cell lines have demonstrated susceptibility to IPNV, the viral titers produced by these cell lines are allegedly low.

In the invention to be presented below, an immortal cell line (GF-1 cell line) derived from the fin tissue of grouper Epinephelus coioides (Hamilton) will be introduced: The GF-1 cell line of the present invention is susceptible to various viruses, particularly fish nodavirus such as NNV. In addition to the viruses that are commonly found in groupers, the GF-1 cell line is capable of replicating IPNV in high titers, even though IPNV is not a virus commonly known to cause diseases in groupers. Therefore, both NNV and IPNV can be mass-produced using the GF-1 cell line, which are suitable for use in antibody and vaccine production to protect fish from viral infections.

### SUMMARY OF THE INVENTION

The present invention provides an antibody which has binding specificity for a virus, the virus is replicated in and harvested from an immortal cell line from Epinephelus coioides having an ATCC accession No. PTA-859. The antibody is either a polyclonal antibody or a monoclonal antibody. In particular, the antibody described in this invention is either an antibody against nervous necrosis virus (NNV) or a virus against infectious pancreatic necrosis virus (IPNV).

To prepare a polyclonal antibody against NNV or IPNV, an effective amount of the purified virus is first injected into an antibody-producing animal to stimulate an immunoresponse. The serum from the animal is then collect and further purified, preferably by affinity column chromatography. The preferred antibody-producing animal is mouse or rabbit.

To prepare monoclonal antibody from against NNV or PNV, an effective amount of the virus is injected into an antibody-producing animal to stimulate an immunoresponse. The splenic cells of the animal are harvested from the antibody-producing animal. The splenic cells are fused with mouse myeloma cells to form fused cells, which is then cultured to form hybridoma cells. The preferred antibody-producing animal is mouse.

The monoclonal antibody is collected from the culture medium of hybridoma cells or from mouse ascites after said hybridoma cells are injected intraperitoneally to a mouse ascites.

The present invention also provides a method for detecting NNV or IPNV in susceptible fish using immunoassay. The preferred immunoassays include basic sandwich immunoassay, triple sandwich immunoassay, or immunochromatographic assay, which can be used to both detect and quantify the virus.

All of the above immunoassay methods require at least two primary antibodies (namely, the first antibody and second antibody), which are both against the viral antigens. These antibodies are preferably polyclonal antibodies obtained by sensitizing antibody-producing animals with purified virus produced from the immortal cell line from Epinephelus coioides having an ATCC accession No. PTA-859. Preferably, the antibodies are purified by affinity column chromatography. The two primary antibodies can be produced by the same or different animal species. The preferred animal species for producing antibodies are rabbits. Optionally, a secondary antibody against the animal species for producing one of the primary antibodies can be added to the assay. In that case, the secondary antibody is conjugated with the detection agent and the primary antibody that is not bound to the solid carrier is not conjugated with a detection agent. A primary antibody is an antibody raised against an antigen, which in this case is the virus. The secondary antibody is an antibody against the immunoglobulin of a primary antibody-producing species (such as goat anti-rabbit IgG).

The basic immunoassay method includes the steps of: (1) providing a fish tissue extract containing the virus; (2) contacting the tissue extract with a first antibody for said virus to form a first antigen-antibody complex; (3) contacting the antigen-antibody complex with a second antibody for said virus to form a second antigen-antibody complex; and (4) detecting the presence of the virus by adding a reagent suitable for detecting or amplifying said detection agent. If the first antibody is bound to a solid carrier, the second antibody must be labelled with a detection agent, and vice versa. Also, between step (2) and (3), the antigen-antibody complex is preferably washed to separate the unbound substance from the antigen-antibody complex. Also, the solid carrier include nitrocellulose membrane, polyester membrane, nylon membrane, filter paper, agarose gel, plastic beads, polyethylene, polystyrene, and polypropylene. If the detection agent is horseradish peroxidase, the reagent (substrate) is preferably tetramethyl benzidine. If the detection agent is alkaline phosphatase, the reagent (substrate) is preferably 5-bromo-4-chloro-3-indoryl phosphate.

The triple sandwich immunoassay method differs from the basic sandwich method as follows: when the two primary antibodies are used, one primary antibody is bound to the solid carrier, the other does not need to be conjugated to a detection agent. Instead, a secondary antibody against the immunoglobulin (Ig) of the animal species producing the unbounded primary antibody to form a complex against the antibody-antigen-antibody complex. The secondary antibody is labeled with a detection agent.

The immunochromatographic assay differs from the basic and triple sandwich immunoassays as follows: (1) the first antibody (i.e., the antibody which is in touch with the fish tissue extract first) must be labelled with a detection agent. The preferred detection agent is color particles, such as gold, silver, blue latex, and selenium. (2) The seond antibody must be bound to a solid carrier such as nitrocellulose membrane.

Furthermore, if the virus is nervous necrosis virus (NNV), the fish tissue is preferably the brain or spinal tissue of parrotfish, sea bass, turbot, grouper, stripped jack, tiger puffer, berfin flounder, halibut, barramundi, and spotted wolffish. If the virus is infectious pancreatic necrosis virus (IPNV), the fish tissue is preferably the pancreatic or intestinal tissue from trout, salmon, carp, perch, pike, and eel.

### BRIEF DESCRIPTION OF THE DRAWINGS

FIG. 1 shows the morphology of the GF-I cells observed under an inverted microscope. (A) A semi-confluent monolayer where both fibroblast-like and epithelial cells co-existed, and (B) a confluent monolayer of GF-1 cells at subculture 80 where fibroblast-like cells were the predominant cells. In the figure, an arrowhead indicates fibroblast-like cells. Similarly, an arrow indicates epitheloid cells. Bar=10 μm

FIG. 2 shows the chromosome number distribution of the GF-1 cells at (A) subculture 50, and (B) subculture 80.

FIG. 3 shows the effect of fetal bovine serum (FBS) on the growth rate of GF-1 cells at (A) subculture 50, and (B) subculture 80.

FIG. 4 shows the effect of temperature on the growth rate of GF-1 cells at subculture 80.

FIG. 5 shows the cytopathic effects (CPE) of GF-1 cells at subculture 80 after infection by (A) IPNV AB strain, (B) IPNV SP strain, (C) IPNV VR299 strain, (D) IPNV EVE strain, (E) HCRV, (F) fish nodavirus GNNV isolate, and (G) HEVF, as compared with (H) Uninfected GF-1 cells.

FIG. 6 shows the agarose gel electrophoresis of the product by RT-PCR amplification using a pair of primers (SEQ ID NO: 1 and SEQ ID NO:2) specific to the target region T4 of fish nodavirus SJNNV. Lane 1, PCR product from GNNV-infected GF-1 cells; lane 2, PCR product from non-infected GF-1 cells. M: pGEM marker.

FIG. 7 is an electron micrograph of GNNV-infected GF-1 cells. Inclusion bodies (indicated by arrowhead) and numerous non-enveloped viral particles are shown in the cytoplasm. I: an inclusion body filled with viral particles. M: mitochondria, N: nucleus. Arrowhead indicates the viral particle. Bar=1 μm.

### DETAILED DESCRIPTION OF THE INVENTION

The present invention provides polyclonal and monoclonal antibodies against NNV or IPNV. The antibodies are particularly useful for the detection of viral infection in susceptible fish. Susceptible fish are those species of fish of which a particular virus is a pathogen.

VNN disease is caused by nervous necrosis virus (NNV), a kind of fish nodavirus. It has been found in many marine fish, such as parrotfish, sea bass, turbot, grouper, stripped jack, tiger puffer, berfin flounder, halibut, barramundi, and spotted wolffish.

IPN disease caused by infectious pancreatic necrosis virus (IPNV). It is a lethal disease in both hatchery-reared juvenile salmonids and nonsalmonid fish. Salmonid fish include, but are limited to, pacific salmon in general (Oncorhynchus sp.), such as rainbow trout (Oncorhynchus mykiss), brook trout (Salvelinus fontinolis), chinook salmon (Oncorhynchus tshawytscha), coho salmon (Oncorhynchus kisutch), sockeye salmon (Oncorhynchus nerca) and Atlantic salmon (Salmo salar).

The general practice in dealing with disease outbreak in fish is by destroying the infected stocks and decontaminating the hatchery facilities because there is no effective methods for controlling viral diseases in fish. Therefore, an effective and early detection of the virus in fish can have immense effects on containing the wide-spread of the virus.

There are many ways to detect viral infection in fish. The most cost-effective way is probably by immunoassays. That is because the principles and practice of immunoassays are well developed. See e.g., Price C. et al. Ed., “Principles and Practice of Immunoassay” (1991), M Stockton Press, Macmillan Publishers Ltd., which is herein incorporated by reference.

The preferred immunoassays for detecting fish samples infected with NNV or IPNV include basic sandwich immunoassays, triple sandwich immunoassays, and immunochromatographic assays.

In the above-mentioned immunoassays, at least two primary antibodies (namely, the first antibody and second antibody) are required, which are both against viral antigens. Preferably, these antibodies are polyclonal antibodies obtained by sensitizing antibody-producing animals with the virus. The antibodies are preferably purified by affinity column chromatography. The two primary antibodies can be produced by the same or different animal species. Examples of animal species for producing antibodies include rabbits, cows, and goats. Optionally, a secondary antibody against the animal species for producing one of the primary antibodies can be added to the assay. A primary antibody is an antibody raised against an antigen. The secondary antibody is an antibody against the immunoglobulin of a primary antibody producing species (such as goat anti-rabbit Ig).

In the basic sandwich assay, only the two primary antibodies are required, in which one is bound to a solid carrier and the other is labelled with a detection agent. The solid carrier can be nitrocellulose (or nitrocellulose derivative) membrane, nylon membrane, polyester membrane, filter paper, agarose or sphedex gel, plastic beads, polyethylene, polystyrene, polypropylene, etc. The preferred solid carrier for a basic sandwich assay or a triple sandwich assay is a polyethylene, polystyrene, or polypropylene plate or well. The preferred solid carrier for the immunochromatographic assay is a nitrocellulose membrane.

The detection agent can be an enzymatic marker (such as alkaline phosphatase or horseradish peroxidase), a fluorescent or luminescent agent (such as fluorescein, rhodamine, or europium, luminol, or acridium), a radioisotope labelling (such as I¹²⁵), or a color particle (such as gold, silver, blue-latex, or selenium).

The triple sandwich assay requires the combined use of two primary antibodies (namely, the first antibody and the second antibody), both against the virus (i.e., NNV or IPNV), in which only one primary antibody is required to be bound to a solid carrier, and one secondary antibody against the IgG of the animal species producing the unbounded primary antibody to form a complex against the antibody-antigen-antibody complex. The secondary antibody is labeled with a detection agent.

Both the basic and triple immunoassay methods cover not only “forward” assays, but also “simultaneous” and “reverse” assays. A “forward” assay involves the contact of the specimen first with an antibody which is bound to a solid carrier, followed by contacting the bound antigen-antibody complex with another antibody which is labelled with a detection agent. A simultaneous assay involves a single incubation step where the antibody bound to the solid carrier and the labelled antibody are both contacted with the testing sample at the same time. A “reverse” assay involves the stepwise addition of first the unbound antibody and the specimen to form an “antigen-antibody” complex, followed by the addition of the “antigen-antibody” complex to the antibody which is bound to the solid carrier.

The immunochromatographic assay also requires the combined use of the two primary antibodies against the virus. Contrasting to the basic and triple sandwich assays, the first antibody (i.e., the antibody that is first in touch with the fish tissue extract) is labelled with color particles. The second antibody is bound to a solid carrier, preferably a nitrocellulose membrane.

There are two regions in the immunochromatographic assay device, which are: a sample addition region and a result viewing region. The first antibody which is labelled with color particles is in contact with the sample at the sample addition region. The second antibody which is bound to the solid carrier is placed within the result viewing region. When the assay is performed, the sample (i.e., fish tissue extract) is in contact with the first antibody at the sample addition region. If the sample contains the viral antigen, a first-antibody-antigen complex is formed and the complex migrates to the result viewing region where the second antibody is located, where an antibody-antigen-antibody complex is further formed. The antibody-antigen-antibody complex is stationed at the result viewing region, which can be observed by the color of the color particles in plain view. If the sample does not contain any viral antigen, the first antibody will run pass the second antibody and not station at the result viewing region.

Optionally, a secondary antibody against the animal species for producing the first antibody can be added and/or bound to the solid carrier at near the end of the chromatographic strip opposite to the sample addition site. This secondary antibody is used as a control for capturing the unbound color particles at the end of the chromatographic run. Thus, if the fish tissue specimen does not contain viral antigens, the color particles labelled with the first antibody will run through the second antibody without binding to it because no antibody-antigen-antibody complex is formed. However, because the secondary antibody is against the immunoglobulin of the first antibody-producing animal, it will bind to the first antibody when it runs by regardless whether the first antibody has form a complex with the sample. The binding between the first antibody and the secondary antibody shows the end of the immunochromatographic run.

The GF-1 cell line of the present invention presents a great opportunity for antibody development against NNV and/or IPNV. Although GF-1 is an immortal cell line derived from the fin tissue of grouper Epinephelus coioides (the fin tissue is not the target tissue for NNV in grouper; and grouper is not a susceptible fish for IPNV), the GF-1 cell line can produce both NNV and IPNV in great quantity. In fact, the titers of IPNV in GF-1 are about 100 times higher than those in CHSE-214 cell line, a well-known cell line derived from chinook salmon embryo which is by far the best known cell line for production of IPNV. Thus, using GF-1 cell line to produce NNV or IPNV opens the door for the production of antibody.

The following examples are illustrative, and should not be viewed as limiting the scope of the present invention. Reasonable variations, such as those occur to reasonable artisan, can be made herein without departing from the scope of the present invention.

### EXAMPLE 1

### ESTABLISHMENT OF THE GF-1 CELL LINE

A vital sample of the immortal cell line derived from the fin tissue of grouper Epinephelus coioides, the GF-1 cell line, was deposited at the American Type Culture Collection (ATCC), 10801 University Blvd., Manassas, Va. 20110-2209, on Oct. 20, 1999, in compliance with the provisions of the Budapest Treaty for the International Recognition of the Deposit of Microorganisms for the Purpose of Patent Procedure. The assigned deposit number for this cell line is ATCC No. PTA-859. The viability of the GF-1 cell line was tested and confirmed on Nov. 1, 1999. The strain will be made available if a patent office signatory to the Budapest Treaty certifies one's right to receive, or if a U.S. Patent is issued citing the strain, and ATCC is instructed by the United States Patent & Trademark Office or the depositor to release the strain.

The GF-1 cell line was established and maintained as follows:

(1) Primary Culture

A grouper (Epinephelus coioides, Hamilton) weighing 1 kg was used for the establishment of the primary culture. The fish was dipped in 5% chlorex for 5 min, and then wiped with 70% alcohol. The fin was dissected from the body, and washed three times in a washing medium (containing L15 plus 400 IU/ml of penicillin, 400 μg/ml of streptomycin and 10 μg/ml of fungizone). After washing, the fin tissue was minced with scissors and then placed into 0.25% trypsin solution (0.25% trypsin and 0.2% EDTA in phosphate-buffered saline[PBS]). The tissue fragments in trypsin solution were slowly agitated with a magnetic stirrer at 4° C. At 30 min intervals, cells released from the tissue fragment were collected by centrifugation. Next, cells were re-suspended in a complete medium (containing L15 plus 20% of fetal bovine serum [FBS], 100 IU/ml of penicillin, 100 μg/ml of streptomycin, and 2.5 μg/ml of fungizone), transferred into a 25 cm² tissue culture flask and, finally, cultured at 28° C.

(2) Subculture and Maintenance

When the confluent monolayer of cells had formed in the primary culture, cells were dislodged from the flask surface by treating with 0.1% trypsin solution (containing 0.1% of trypsin and 0.2% of EDTA in PBS). The released cells were then transferred into two new flasks containing fresh L15 medium plus 20% of FBS. Cells were subcultured at a split ratio of 1:2. For the first ten subcultures of the GF-1 cells, a conditioned medium consisting of 50% old and 50% fresh medium was used. The concentration of FBS in the maintaining L15 medium was 10% for subcultures 11-70, and decreased to 5% after subcultures 70. Also, during the first twenty passages, GF-1 cells were subcultured at a 9-day interval. For the next 21^{th}-70^{th} passages, the GF-1 cells were subcultured at a 5-day interval. After 71 passages, GF-1 cells were subcultured at a 3 -day interval.

(3) Test for Mycoplasma Contamination in the GF-1 Cell line

The GF-1 cell line was propagated for three transfers in antibiotic-free L15-10% FBS and tested for the presence of bacteria, fungi, and mycoplasma. A mycoplasma stain kit (Flow Laboratories, U.S.A.) was used for mycoplasma testing.

(4) Test for the Viability of the GF-1 Cell Line

The viability of the GF-1 cell line was tested by first removing the cells from the flask. Then, the cells were separated from the medium by centrifugation, and re-suspended in a freezing medium consisting of 10% dimethyl sulfoxide (DMSO) and 90% FBS. Ampules (NUNC, Denmark) containing 5×10⁶ cells/ml /ampule were held at −20° C. for one hour, followed by staying at −70° C. overnight before being transferred to liquid nitrogen (−176° C.). After one month and one year, the ampules were thawed in a 30° C. water bath. The cells were separated from the freezing medium by centrifugation. The cells were re-suspended in L15-10% FBS. The viable cells were determined by trypan blue staining. The number of cells was counted using a hemacytometer. The thawed cells were re-seeded into a 25 cm² flask for further observation.

(5) Chromosome Number Distribution

The distribution of the chromosome numbers in GF-1 cells at subculture 50 and subculture 80 were studied using semi-confluent and actively growing cells. Cells were pre-treated with 0.1 μg/ml Colcemid (Gibco, Grand Island, N.Y.) for 5 hours at 28° C. before being dislodged with 0.1% of trypsin solution. After centrifugation at 1000 g for 10 min, the cells were re-suspended in a hypotonic solution (containing 8 parts of distilled water and 1 part of PBS) for 30 min. The cells were then partially fixed by adding several drops of Carnoy fixative (containing 1 part of Glacial acetic acid and 3 parts of 100% methanol). The partially fixed cells were further centrifuged at 800 g for 10 min at 4° C. The supernatant was discarded, and the cells were fixed in fresh, cold Camoy fixative for 20 min. The suspension of fixed cells was dropped onto a 76×26 mm slide. The slide was air-dried and the cells were stained with 0.4% Giemsa stain (Sigma, St. Louis, Mo., USA) for 30 min. The chromosome numbers were observed and counted under an Olympus Vanox microscope.

(6) Plating Efficiency

The plating efficiency of the GF-1 cells was estimated at subcultures 50 and 80. Cells were seeded into a 25 cm² flask at a density of 100 cells per flask. Following 15 days of incubation, the medium was removed and the cell colonies were fixed with 70% ethanol and stained with 0.4% Giemsa. The colonies in each flask were then counted using an Olympus IM inverted microscope. Carp fin (CF), black porgy spleen (BPS-1), tilapia ovary (TO-2) and eel kidney (EK) cell lines were plated the same way as the GF-1 cell line for comparison purpose.

(7) Effects of FBS Concentration and Temperature on the Growth of the GF-1 cells

The effects of the concentration of FBS on GF-1 cell growth were determined at subcultures 50 and 80. Two replicates were prepared for each FBS concentration. At selected intervals, two flasks were withdrawn from each concentration of FBS, and the mean number of cells was counted.

To determine the effects of temperature on the growth of GF-1 cells at subculture 80, replicated cell cultures in 25 cm² flasks containing L15-10% FBS were incubated at 18° C., 28° C. and 35° C. The mean number of the GF-1 cells from two replicated flasks at each temperature was counted at selected intervals.

Results:

Primary Culture and Subculture of the GF-1 Cells

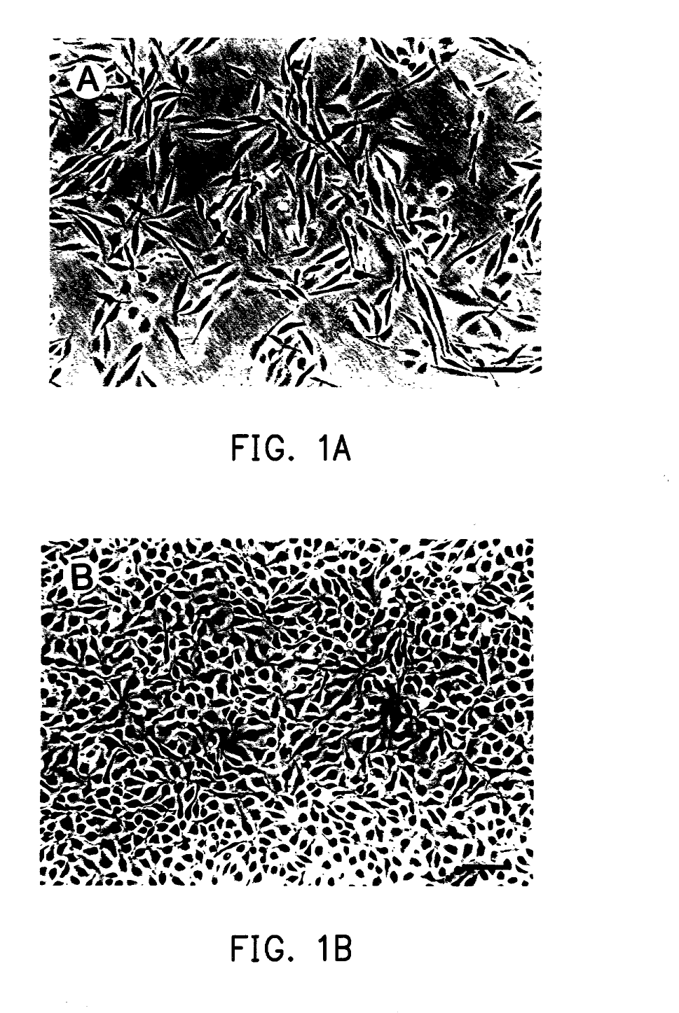

A monolayer of cells was formed in the primary culture approximately two weeks after the implantation. Fibroblast-like cells and epitheloid cells co-exist in the cell population (FIG. 1). The GF-1 cells have been successfully subcultured for more than 160 times since 1995, subsequently becoming a continuous cell line.

The GF-1 cells were subcultured at 9-day intervals in L15-20% of FBS during the first twenty subcultures, at 5-day intervals in L15-10% of FBS during the 21^{st}-70^{th} subcultures, and at 3-day intervals in L15-5% of FBS since subculture 71. Contact inhibition of the GF-1 cells was found in cultures before subculture 50, and gradually decreased between subculture 51 and 80.

The viability of the GF-1 cells at subculture 80 after one year and one month was 73%. The re-seeded cells grew readily when incubated at 28° C. in L15-5% of FBS.

Chromosome Number

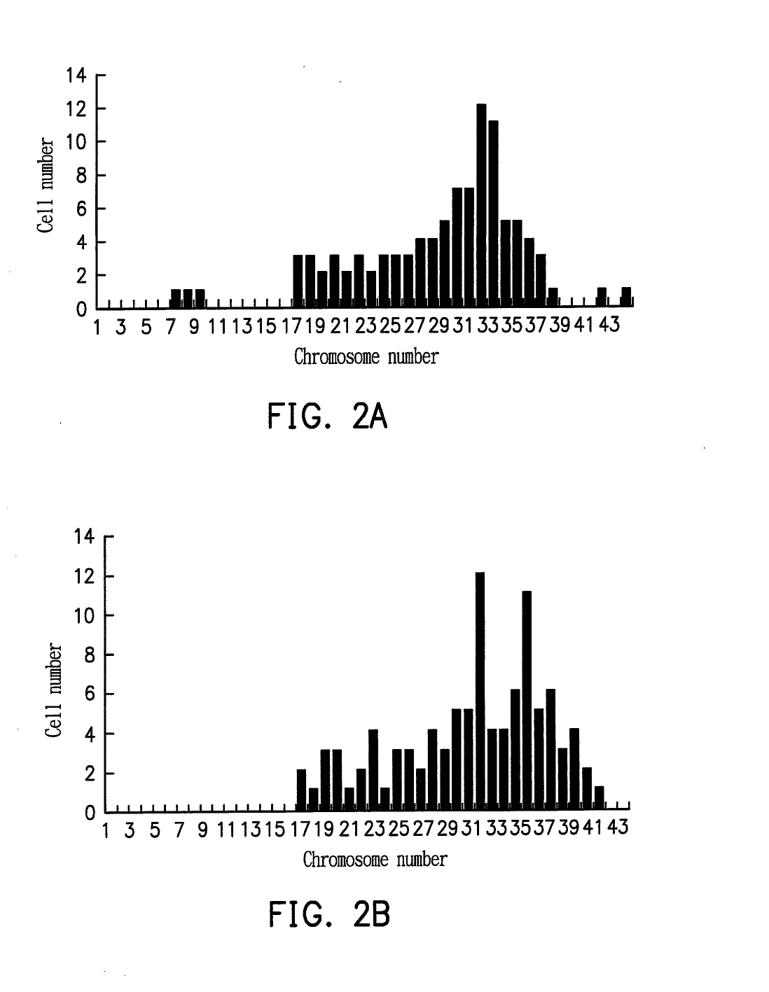

The chromosome number of the GF-1 cells at subculture 50 was distributed between 7 and 44 with the mode set at 32 (FIG. 2A). The chromosome number of the GF-1 cells at subculture 80 was distributed between 17 and 42 in 100 cells examined, and had a bimodal distribution with modes set at 32 and 36 (FIG. 2B). Both micro- and macro-chromosomes were found in metaphase-arrested cells.

Plating Efficiency

The plating efficiency of the GF-1 cells seeded at a density of 100 cells/ flask was 21% at subculture 50 which increased to 80% at subculture 80. In comparison, the plating efficiencies of CF, BPS-1, TO-2 and EK cell lines seeded at a density of 100 cells/flask were 22%, 13%, 48%, and 63%, respectively. The increase in plating efficiency in GF-1 cells suggests the occurrence of transformation during subcultures 50-80.

Effects of FBS Concentration and Temperature on the Growth of the GF-1 cells

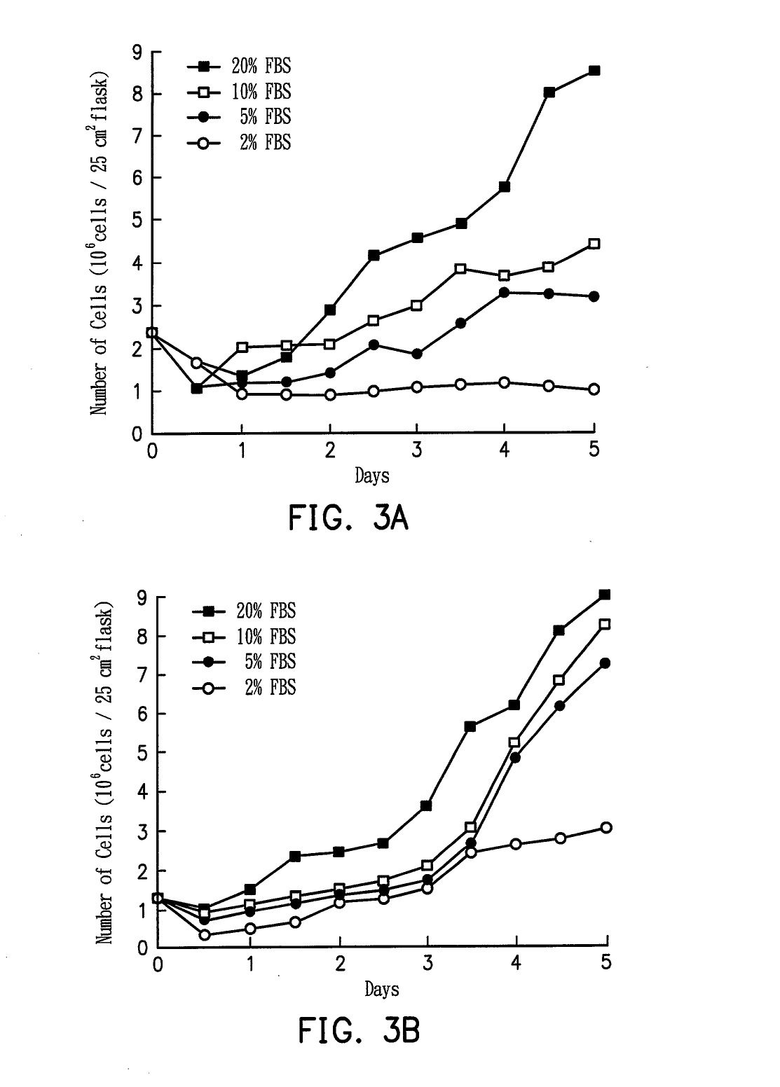

FIG. 3 illustrates the effects of FBS concentration on the growth of the GF-1 cells at subcultures 50 and 80. The growth of the GF-1 cells at both subcultures 50 and 80 corresponded to the concentration of FBS, i.e., the higher the FBS concentration, the greater the growth of cells. However, when the growth rates of the GF-1 cells at subcultures 50 and 80 were compared, the GF-1 cells at subculture 80 demonstrated a much greater growth potential than those at subculture 50, especially when the FBS concentrations were at 2%, 5%, and 10%. For example, at day 4 of the cell cultures containing 10% of FBS, the GF-1 cells at subculture 50 have 3.5×10⁶ cells/25 cm² flask, whereas the GF-1 cells at subculture 80 have 5.0×10⁶ cells/25 cm² flask. These results suggest that the requirement of FBS for cell growth decreased at subculture 80, which is an indication that the transformation of cells had occurred during the period from subculture 50 to subculture 80.

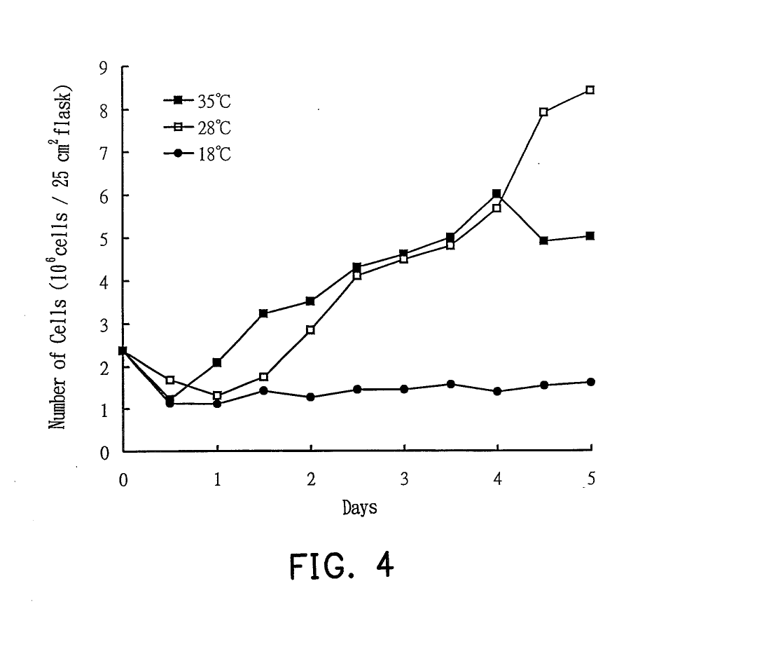

FIG. 4 illustrates the effect of temperature on the growth of the GF-1 cells at subculture 80. The results show that the GF-1 cells grew well at 28° C. and 35° C. However, the growth of the GF-1 cells cultured at 35° C. started to decline at day 4, suggesting that maintaining the cell culture at 35° C. may have long-term effects on cell growth. The GF-1 cells did not grow well at 18° C.

### EXAMPLE 2

### METHODS FOR PRODUCING VIRUSES USING THE GF-1 CELL LINE AND METHODS FOR DETECTING THE VIRUSES IN THE CELL LINE

(1) Test for Susceptibility of the GF-1 Cells to Aquatic Viruses

Infectious pancreatic necrosis virus (IPNV, strain AB, SP, VR299 and EVE), hard clam reovirus (HCRV), eel herpes virus Formosa (EHVF) and nervous necrosis virus (NNV, GNNV isolate) were used to infect the GF-1 cells at subculture 80. The susceptibility to GNNV was also examined in BGF-1 cell line, which was derived from the fin of the banded grouper Epinephelus awoara.

Each of the monolayer GF-1 cells was inoculated with 0.5 ml of various aquatic virus with titer of 10³ TCID₅₀/0.1 ml. After a 30-min adsorption period, the cells from each flask were washed three times with PBS, followed by the addition of 5 ml of L15-2% FBS to each flask. The flasks were then incubated separately at 20° C. and 28° C. The supernatants of culture cells were collected and titrated for 6 days post viral infection.

(2) Multiplication and Purification of Aquatic Viruses in the GF-1 Cell line

Viral isolate was inoculated at an MOI (multiplicity of infection) of 0.01 into the GF-1 cell line. When CPE appeared, the GF-1 cells were scraped into the medium and the cell debris was pelleted at 10000×g for 30 min (the first pellet). The supernatant was transferred to a bottle and polyethylene glycol (PEG, molecular weight 20000) and NaCl were added to reach a final concentration of 5 % and 2.2% separately. The supernatant was then stirred for 4-6 hours at 4° C., and the virus particles were pelleted by centrifugation at 10000×g for 1 hour (the second pellet). The first pellet and the second pellet were re-suspended in a small amount of TNE buffer (0.1M Tris, 0.1M NaCl, 1 mM EDTA, pH 7.3), to which an equal volume of Freon 113 was added. The mixture was shaken vigorously for 5 min, and the emulsion was separated into the Freon and aqueous phase by centrifugation at 3000×g 10 min. The aqueous phase was collected, layered on a preformed 10-40 % (w/w) CsCl gradient, and centrifuged at 160000×g for 20 hours. The visible virus band was collected, diluted with 10 ml of TNE buffer, and pelleted again by centrifugation at 150,000 g for 1 hours. The final pellet was resuspended in a small volume of TE buffer (0.1 M Tris, 1 mM EDTA, pH 7.3).

(3) Detection of Aquatic Viruses in the GF-1 Cell Line

In general, when a virus infects a cell line which is susceptible to the virus, a CPE of the cell culture can be observed within a couple of days after the infection. The appearance of CPE serves as evidence that the virus has successfully infected and multiplied in the cell line. The viral infection in the cell line can be further confirmed using an electron microscopic technique which is described as follows: The virus-infected cells were fixed in 2.5% glutaraldehyde in 0.1M of phosphate buffer at pH 7.4 and post-fixed in 1% of osmium tetraoxide. The cells were ultrathin sectioned. The ultrathin sections were stained with uranyl acetate-lead citrate and examined under a Hitachi H-600A electron microscope. The viral particles should appear as homogeneous, spherical particles in the cytoplasm of the cells.

There are also four methods that are directed to specific detection of NNV in the GF-1 cell line:

(A) Detection of NNV in the GF-1 cells by Polymerase Chain Reaction (PCR) Amplification

A PCR amplification method was used to confirm that the GF-1 cells are able to proliferate NNV. The method required that the viral RNA be extracted from the supernatant of the NNV-infected cells after CPE appeared using a Rneasy™ mini kit (QIAGEN). For reverse transcription, extracted viral RNA was incubated at 42° C. for 30 min in 40 μl of 2.5 X PCR buffer (25 mM of Tris-HCl, pH 8.8, 3.75 mM of MgCl₂, 125 mM of KCl, and 0.25% of Triton X-100) containing 2 U of MMLV reverse transcriptase (Promega), 0.4 U of RNsin (Promega), 0.25 mM of dNTP, and 0.5 μM of the reverse primer R3 (5′CGAGTCAACACGGGTGAAGA 3′) (SEQ ID NO. 1). Following the cDNA synthesis, 40 μl of the cDNA mixture were diluted 2.5-fold with diethyl pyrocarbonate (DEPC)-treated H₂O (containing 0.025 U of DNA polymerase [Biometra], 0.1 mM of dNTP and 0.5 μM of the forward primer F2 [5′CGTGTCAGTCATGTGTCGCT 3′] [SEQ ID NO.2]), and incubated in an automatic thermal cycler (TouchDown™ thermal cycler, Hybaid company). The target region for the primer set (F2, R3) is T4 (400 bp). The PCR products corresponding to T2 and T4 were amplified from the nucleic acids of NNV-infected GF-1 cells.

(B) Detection of NNV in the GF-1 Cells by Western Immunoblot

A western immunoblot method was used to specifically detect the NNV proteins. The viral sample was prepared as follows: NNV was inoculated into the GF-1 cells and incubated at 20-32° C. After 5 days of incubation, the NNV-infected cells were pelleted by centrifugation at 1000 g for 10 min. The cell pellets were loaded onto a 10% SDS-polyacrylamide gel. After electrophoresis, the proteins were blotted to an immobilon-P transfer membrane (Millipore), which was then soaked in a 3% skim milk tris buffered saline (TBS) for 1 hr. The membrane was then incubated with an antiserum against NNV for 1 hr at room temperature, washed with TBS, reacted with a peroxidase-conjugate goat system for 1 hr, and stained with a substrate containing 6 mg of 4 -chloronaphthol in 20 ml of methanol and 60 μl of H₂O₂ in 100 ml of TBS.

(C) Detection of NNV in the GF-1 Cells by Enzyme-Linked Immunoabsorbent Assay (ELISA)

ELISA is an immunological method which uses an enzyme-labeled immunoreactant (antigen or antibody) and an immunosorbent (antigen or antibody bound to a solid) to identify specific serum or tissue antibodies or antigens. The ELISA test was conducted as follows: an effective amount of purified NNV proteins was coated onto a microtiter plate at 4° C. overnight. Then, 3% of bovine serum albumin (BSA) was added to the plate (used as blocking agent) and incubated at 37° C. for 1 hr. The plate was then washed 3 times with buffer. Next, a diluted rabbit anti-NNV serum was added to the plate and incubated at 37° C. for 1 hr. This was followed by the addition of goat anti-rabbit IgG-horseradish peroxidase serum at 37° C. for 1 hr and 3,3′,5,5′-tetramethyl benzidine was added for color development. The color reaction was stopped with 1 N H₂SO₄. The optical density of the wells in the microtiter plate was measured at 450 nm with an ELISA reader (Dynatech MR 5000).

(D) Detection of NNV in the GF-1 Cells by Immunofluorescent Staining

To detect the virus that proliferated in the GF-1 cells, cell cultures were fixed by 10% formalin for 12 hrs after viral infection. The fixed cell cultures were treated with 0.2% of Triton X-100 and washed with PBST (phosphate buffer with 0.05% Tween 20). The Triton-treated cell cultures were further washed with 3% of skim milk as blocking agent and then reacted with mouse anti-NNV serum. Finally, the antibody-treated cell cultures were stained with fluorescein isothiocyanate (FITC) conjugated goat anti-mouse antibodies.

Results:

Table 1 summarizes the results of virus susceptibilities of the GF-1 cells to IPNV (AB, SP, VR299, EVE strains), HCRV, EHVF and NNV (GNNV isolate), which were determined by first observing the appearance of CPE in the cells after the viral inoculation, followed by the determination of viral titers (TCID₅₀/ml).

TABLE 1
Viral Susceptibilities of GF-1 Cells at Subculture 80

| | | Initial Viral | | | Virus Yield/ml | |
|---|---|---|---|---|---|---|
| | | Inoculum | CPE | | (TCID₅₀/ml) | |
| Cell line | Virus | (TCID₅₀) | 28° C. | 20° C. | 28° C. | 20° C. |
| GF | IPNV | | | | | |
| | AB | 10³ | — | + | ND | 10^{9.5} |
| | SP | 10³ | — | + | ND | 10^{10.8} |
| | VR299 | 10³ | — | + | ND | 10^{9.8} |
| | EVE | 10³ | — | + | ND | 10^{9.6} |
| | HCRV | 10³ | — | + | ND | 10^{11.0} |
| | EHVF | 10³ | + | + | 10^{8.1} | 10^{7.0} |
| | GNNV | 10³ | + | — | 10^{8.3} | ND |

ND: Not done.
+: Cytopathic effect (CPE) was observed.

As shown in Table 1, for the IPNV strains and HCRV, CPE appear only when the cells are incubated at 20° C. For EHVF, CPE appears at both 20° C. and 28° C. However, for GNNV, CPE appears at 28° C. The yields of the viruses in GF-1 cells at subculture 80, which are ranged between 10^{7.0} (as for EHVF at 20° C.) and 10^{11.0} (as for HCRV at 20° C.), are extremely high.

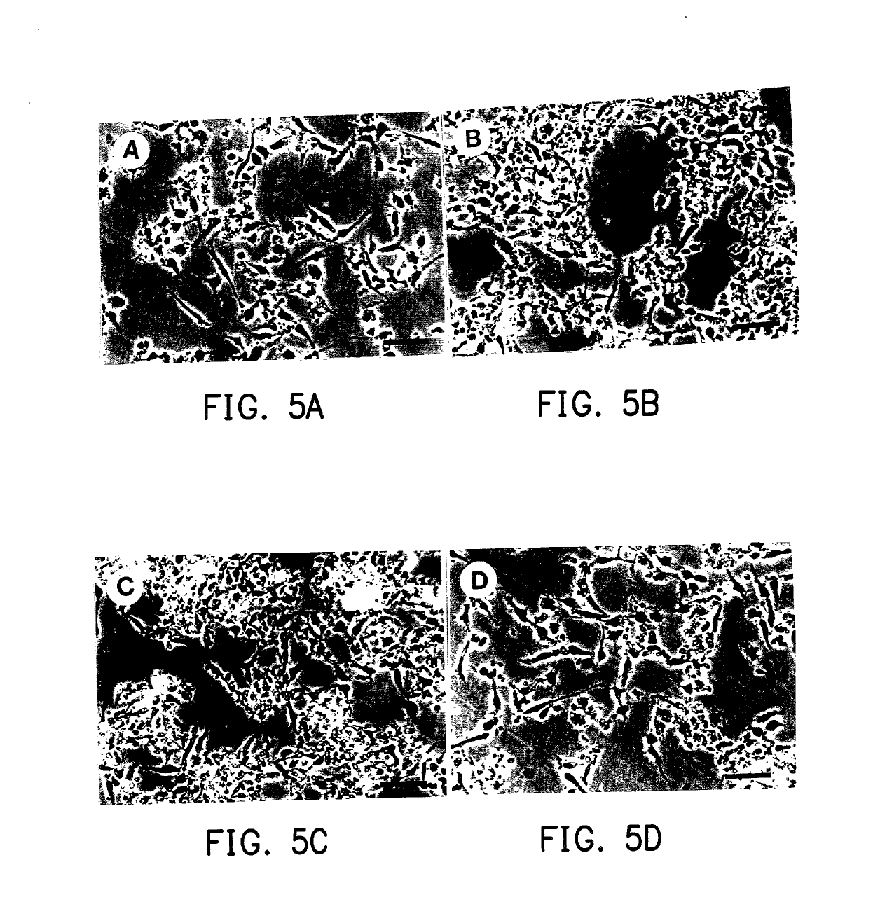

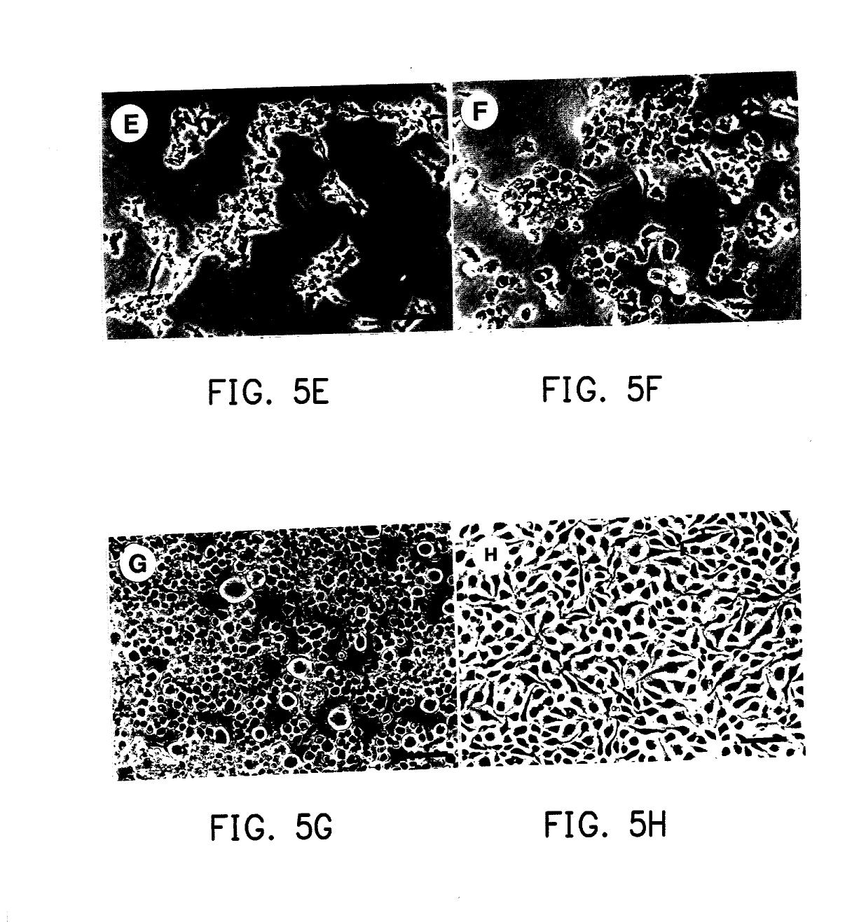

Typically, for an aquatic virus such as GNNV, CPE began at the 3rd day of infection when some rounded, granular, refractile cells began to appear in the cell culture (FIG. 5). Soon more and more cells became round and swollen. The swollen cells became larger and finally started to detach from the cell culture and float in the culture media. Most of the detached cells were completely disintegrated. The culture fluid from cell culture showing CPE could transmit other GF-1 cells. This experiment also tested the susceptibility of BGF-1 cell line (derived from the fin of the banded grouper Epinephelus awoara) to GNNV. The results showed that no CPE was found after the viral infection.

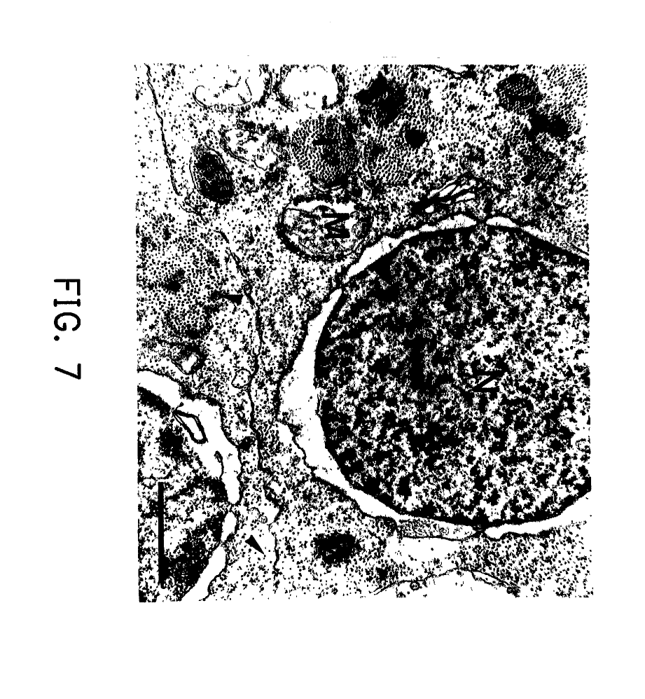

Typically, for an aquatic virus such as GNNV, the virus could be observed in the cytoplasm of the GF-1 cells under electron microscope as numerous non-enveloped, homogeneous, spherical to icosahedral particles with diameter of 20-25 nm (FIG. 7). Some of the viral particles were included in the inclusion bodies and the others could be found in the cytoplasm (FIG. 7). The isolated viral particles could be further purified by CsCl density gradient centrifugation. Using GNNV as an example, the purified virus was a non-enveloped icosahedral virion particle with the diameter of 20-25 nm. The buoyant density of GNNV in CsCl was 1.34 g/cm³.

As for IPNV, the virus can be concentrated by polyethylene glycol-NaCl precipitation and purified by Freon extraction and isopycnic CsCl gradient centrifugation, as described by Bootland et al., J. Fish Diseases (1995) 18:449-458. The buoyant density of IPNV in CsCl is 1.33 g/cm³.

In addition to the findings of CPE in the GF-1 cells, the existence of an aquatic virus in the GF-1 cells and the capability of the cells to multiply the virus can be further confirmed by four methods: (1) the PCR method; (2) the Western immunoblot method; (3) the ELISA method; and (4) the immunofluorescent staining method.

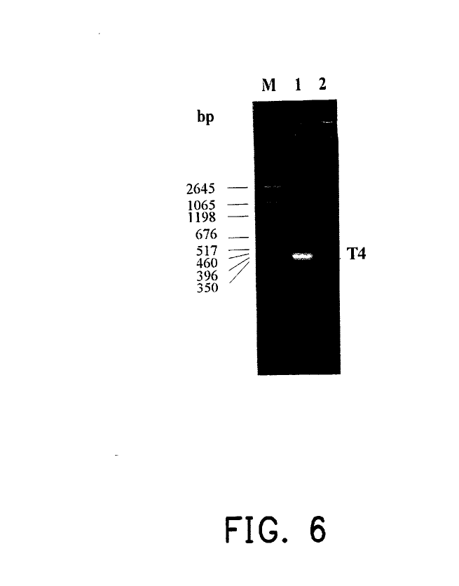

Using GNNV as an example, the PCR method could be accomplished by choosing a pair of primers, i.e., R3 (SEQ ID NO.1) and F2 (SEQ ID NO.2), for PCR amplification. The target fragment T4 exists in fish nodavirus. Therefore, the PCR method using F2 and R3 was specific to fish nodavirus, not just GNNV. The results of the PCR study showed that GNNV could be replicated in the GF-1 cells and released into the supernatant of culture cells (FIG. 6).

The Western immunoblot using mouse anti-GNNV serum demonstrated that viral proteins were present in the GNNV-infected cells cultured at 20-32° C., suggesting that the viral mRNA could be successfully translated into viral polypeptides within the host cells when the culture was maintained at 20-32° C.

The ELISA and immunofluorescent staining methods also showed positive reactions with the anti-GNNV serum, indicating that GNNV could be multiplied in the GF-1 cells.

### EXAMPLE 3

### PRODUCTION OF ANTI-NNV ANTIBODIES

(1) Production of Polyclonal Anti-NNV or Anti-IPNV Antibodies

Polyclonal antibodies can be produced in accordance with conventional methods, e.g., by sequential injections of the purified NNV or IPNV immunogen into a suitable animal such as a rabbit. For example, a suitable amount of the NNV immunogen can be injected intravenously, subcutaneously, or intraperitoneally to a rabbit and boosted twice or more at 2 or 3 week intervals. The injection may contain a suitable amount of Freund's complete or incomplete adjuvant, if necessary.

For example, if a rabbit is used for the production of antibody, a trial bleed can be taken after three months of the initial injection (approximately one week after the third injection). The bleed can be taken from the central ear vein of the rabbit. The blood is allowed to clot at 2-8° C. overnight and then centrifuged at 5,000 xg for 15 minutes at room temperature. The supernatant (serum) is collected and tested by an Indirect Fluorescent Assay (IFA).

The volume bleed can be obtained similarly to the trial bleed except that 50 ml is removed from each rabbit. The serum can then be further purified by conventional immunoglobulin purification procedures such as ammonium sulfate precipitation, gel electrophoresis, dialysis, affinity chromatography, and ultrafiltration. Ion exchange, size exclusion hydroxylapatite, or hydrophobic interaction chromatography can be employed, either alone or in combination. Light and heavy chain can be carried out using gel electrophoretic techniques or isoelectric focusing, as well as other techniques known in the art. The preferred antibody purification method is by affinity column chromatography.

An affinity column can be prepared by conjugating the purified NNV or IPNV virus to the affinity gel. The NNV or IPNV is purified according to EXAMPLE 2. Preferably, the NNV or IPNV is pre-treated with heat (especially for NNV) or formalin (especially for IPNV) to inactivate the virus. The suitable heat treatment for NNV is at 60° C. for at least 1 hour. The suitable formalin treatment is by incubating IPNV at 22° C. at 0.5% formalin for about 7 days.

The preferred affinity gel is the prerequilibrated Aminolink gel (manufactured by Pierce). The gel can be mixed with the virus at a ratio of 1-10 mg protein per ml of packed gels, followed by 10-40 μl of 5 M sodium cyanoborohydride in water for each mol of the packed gel, to form a gel slurry. The gel slurry can be incubated at 4° C. overnight (or at least 6 hours) with gentle mixing. After incubation, the gel slurry can be poured into an appropriate size chromatography column and the excess liquid can be drained. The column can then be eluted with two column volumes of a coupling buffer, followed by two column volumes of 1 M Tris-HCl (pH 7.4). The column can then be washed extensively with PBS to remove any unconjugated virus in the column.

The rabbit antibody is loaded onto the packed affinity column. After loading, the antibody against the virus can be eluted with 3 M potassium cyanate (KSCN) in water. The eluted antibody can be dialyzed against PBS until all KSCN is removed.

(2) Production of Monoclonal Antibody Against NNV and IPNV

For the production of monoclonal antibodies, immunizing mice was preferred. Three or four days after the final boost, spleen cells of mice were separated and fused with myeloma cells, e.g., SP2/0-Ag14 myeloma cells (ATCC CRL 1581), in accordance with a conventional method described by Mishell and Shiigi (Selected Mthods in Cellular Immunology, W. H. Freeman & Company, 1980). The spleen cells and the myeloma cells were used in a ratio ranging from 1:1 to 1:4. A fusion-promoting agent, e.g., polyethylene glycol (PEG) 4000, was employed for accelerating the cell fusion. A medium suitable for use in the cell fusion step may be RPMI 1640 (Gibco BRL, Life Technologies, Inc.) and the medium generally contains 10-15% (v/v) fetal bovine serum (FBS).

The fused cells were cultured in a 96-well microculture plates in a growth medium (GM) consisting of the RPMI1640 (Flow Laboratories) with 15% FBS, 2 mM glutamine and 1 mM sodium pyruvate and incubated in 5% CO₂ at 37° C. The cultured fused cells were tested by adding GM which was supplemented with hypoxanthine (0.1 mM), aminopterin (4×10⁻⁴ mM), and thymidine (1.6×10⁻² mM) (HAT) to each well. HAT selection was continued for about 2 weeks by changing half volume of the supernatant of the 96-well microculture plates with GM-HAT at each 3 consecutive days. After seven to ten days, positive hybridomas were cloned by limiting dilution. Further selection of positive clones can be accomplished by using conventional methods, e.g., the limiting dilution technique, the plaque method, spot method, agglutination assay and autoradiographic immunoassay.

Monoclonal antibody isotypes can be determined using an ELISA isotyping kit (SBA Clonotyping System III).

The monoclonal antibodies were collected from directly from hybridoma culture supernatants or mouse ascites. Ascites of selected monoclones were obtained by intraperitoneal (i.p.) injection of 1×10⁶ hybridoma cells into Balb/c mice 10 days after inoculation of Prestain (2,6,10,14-tetramethalpentadecane, Sigma, T-7640).

Western immunoblot technique according to Lo et al., Fish Pathol. (1988) 23:147-154, was used for detection of specificity of monoclonal antibodies against viral polypeptides and to the known viral serotypes. The similar technique was also employed to identify the antigenicity in various isolates of aquatic birnaviruses.

(3) Detection of NNV or IPNV in Fish Samples Using Immunoassays

Detection of NNV or IPNV in susceptible fish can be accomplished by immunoassays, including basic sandwich immunoassays, triple sandwich immunoassays, and immunochromatographic assays.

Fish tissue specimens, such as brain or spinal tissue from fish infected with NNV, or the pancreatic or intestinal tissue from fish infected with IPNV, can be placed in PBS and homogenized with a telfon tissue homogenizer to release the virus in the tissue to the PBS. The homogenate can be centrifuged at 5,000 xg for about 10 minutes to precipitate the tissue debris.

Affinity column purified antibody against NNV or IPNV can be conjugated with Horseradish peroxidase (HRP). The antibody-HRP enzyme conjugates production is according to Nakane et al, (1978): In immunoflorescence and related staining techniques, Knapp, W., Holubar, K., and Wick, G, eds., p215-220, Elsivier/North-Holland Biomedical Press, Amsterdam. Briefly, HRP is first subjected to an oxidation treatment with sodium m-periodate. This oxidation generates an aldehyde group on the carbohydrate side chain. The antibody and oxidized HRP can then be mixed in alkaline pH, allowing the amino group on the antibody to react with the aldehyde group on HRP to form a Schieff base and reduced to a covalent bond between antibody and HRP. The antibody-HRP conjugate can then be purified by gel filtration with a sephacryl-300 column.

The HRP can be conjugated to a primary antibody such as a rabbit antibody against H. pylori, or a secondary antibody such as a goat antibody against rabbit IgG.

Affinity-column purified antibody against NNV or IPNV can be added to a Costar EIA Strip Plate, covered and incubated over night at room temperature. The plate can then be washed once with PBS and blocked with 1% BSA in PBS for 4 hours at room temperature. After BSA solution is removed, an aliquot of the fish tissue extract can be added to the antibody coated microwell strip plate, covered and incubated 2 hours at room temperature. The plate can then be washed 5 times with PBS/Tween 20. Then, an aliquot amount of the antibody conjugated to horseradish peroxidase is added to each microwell, covered and incubated 1 hours at room temperature. Again, the plate was washed 5 times with PBS/Tween 20 and then developed for 10 minutes at room temperature with 0.1 ml of tetramethylbenzidine. Color development can be measured at 450 nm after the reaction is stopped with 0.1 ml of 1N H₂SO₄.

### EXAMPLE 4

### PRODUCTION OF VACCINES

Preparation of vaccine using killed NNV

The vaccine of the present invention is administered as a killed vaccine, which encompasses any methods now known or hereafter developed for killing. The preferable method is by heat treatment. The heat treatment method can be accomplished by heating the purified NNV to a temperature sufficiently to inactivate the virus (such as 70° C.) for a sufficient amount of time (such as for 24 hours). After the heating step has been completed, for intraperitoneal or intramuscular vaccination, the inactivated NNV can be emulsified in Freund's incomplete adjuvant (FIA) using a mixer for several minutes. The vaccine can then be injected into the fish (the primary injection). Booster injections can be given to the fish 30-45 days after the primary injection. Normally, the booster injection consists of about one half of the volume of the vaccine used in the primary injection. The fish then can receive a secondary boost 10 days after the first booster shot is administered. The serum samples from the fish at various time points can be taken for titer determination.

For orally administered vaccine, an enteric coating containing non-toxic polymeric materials can be added to the vaccine. The preferable enteric coating materials are the ones which can resist dissolution at the pH of the stomach but can be dissolved once the material passes from the stomach to the pyloric caecum and intestines. For example, cellulose acetate phthalate, hydroxypropylmethyl cellulose phthalate, carboxymethylethyl cellulose, hydroxypropylmethyl cellulose acetate succinate, cellulose acetate trimellitate, polyvinyl acetate phthalate, EUDAGRIT L-30D and 1100-55, EUDAGRIT L 12.5 and L 100, EUDRAGIT E, RL, RS and NE are among the preferred materials. Additional materials can be used in combination with the enteric coating materials. For instance, plasticizers (such as polyethylene glycol 200, 400, 1000, 4000, 6000, propylene glycol, PVPK-90, glycerin or glycerol, diethyl phthalate, oleic acid, isopropyl myristate, liquid paraffin or mineral oil, triacetin, glycerol monostearate, dibutyl sebacate, triethyl citrate, tributyl citrate, acetylated monoglyceride, dibutyl phthalate, acetyl tributyl citrate, castor oil, and glycerol tributyrate); disintegrants (such as sodium starch glycolate); adjuvants (such as immunostimulants [e.g., beta glucan]); binders (such as starch, polyvinyl pyrrolidone, polyvinyl alcohol); diluents (such as lactose); lubricants (such as magnesium stearate) etc. can all be used with the enteric coatings. For oral administration, fish can receive the vaccine on an every-other-day basis for a total of thirty days. The effects of the vaccines can be monitored by the use of ELISA.

Orally administered vaccine is generally the preferred method of vaccinating fish because it is not limited by the size of the fish that can be handled, and it reduces the stress on the fish associated with immersion and intraperitoneal injection. Furthermore, oral vaccines offer the additional advantages of stimulating the gut-associated lymphoid tissue to a greater extent than does intraperitoneal injection.

The present invention has been described with reference to several preferred embodiments. Other embodiments of the invention will be apparent to those skilled in the art from the consideration of this specification or practice of the invention disclosed herein. It is intended that the specification and examples contained herein be considered as exemplary only, with the true scope and spirit of the invention being indicated by the following claims:

What is claimed is:

1. An antibody having binding specificit for a virus, wherein said virus is replicated in an immortal cell line from Epinephelus coioides having an ATCC accession No. PTA-859.

2. The antibody according to claim 1, wherein said antibody is a polyclonal antibody.

3. The antibody according to claim 1, wherein said antibody is a monoclonal antibody.

4. The antibody according to claim 1, wherein said virus is a nervous necrosis virus (NNV).

5. The antibody according to claim 1, wherein said virus is an infectious pancreatic necrosis virus (IPNV).

6. A method for preparing the antibody according to claim 2 comprising:
injecting an effective amount of said virus to an antibody producing animal to stimulate an immunoresponse; and
collecting and purifying a serum from said animal.

7. The method according to claim 6, wherein said virus is a nervous necrosis virus (NNV).

8. The method according to claim 6, wherein said virus is an infectious pancreatic necrosis virus (IPNV).

9. The method according to claim 6, wherein said suitable animal is mouse or rabbit.

10. A method for preparing antibody according to claim 3 comprising:
injecting an effective amount of said virus to an antibody producing animal to stimulate an immunoresponse;
harvesting splenic cells from said antibody-producing animal;
fusing said splenic cells with mouse myeloma cells; and
culturing said fused cells to form hybridoma cells.

11. The method according to claim 10, wherein said antibody-producing animal is mouse.

12. The method according to claim 10, wherein said monoclonal antibody is collected from the culture medium of hybridoma cells or from mouse ascites after said hybridoma cells are injected intraperitoneally to a mouse ascites.

13. An immunoassay method for detecting said virus according to claim 1 comprising:
providing a fish tissue extract containing said virus;
contacting said tissue extract with a first antibody for said virus to form a first antigen-antibody complex;
contacting said antigen-antibody complex with a second antibody for said virus to form a second antigen-antibody complex; and
detecting the presence of said virus by adding a reagent suitable for detecting or amplifying said detection agent.

14. The immunoassay method according to claim 13, wherein one of said first antibody and second antibody is bound to a solid carrier and the other is labelled with a detection agent.

15. The immunoassay method according to claim 13, further comprising a secondary antibody conjugated with a detection agent; wherein only one of the first antibody or second antibody is bound to a solid carrier.

16. The method according to claim 13, wherein said detection agent is horseradish peroxidase or alkaline phosphatase.

17. The method according to claim 13, wherein when horseradish peroxidase is the detection agent, said reagent is tetramethyl benzidine; and wherein when alkaline phosphatase is the detection agent, said reagent is 5-bromo-4-chloro-3-indoryl phosphate.

18. The method according to claim 13, wherein solid carrier is one selected from the group consisting of nitrocellulose membrane, polyester membrane, nylon membrane, filter paper, agarose gel, plastic beads, polyethylene, polystyrene, and polypropylene.

19. The method according to claim 13, wherein said first antibody is labelled with color particles and the second antibody is bound to a solid carrier.

20. The method according to claim 19, wherein said solid carrier is a nitrocellulose membrane.

21. The method according to claim 19, wherein said color particles comprise at least one selected from the group consisting of gold, silver, blue latex, and selenium.

22. The method according to claim 13, wherein said virus is nervous necrosis virus (NNV).

23. The method according to claim 22, wherein said fish tissue is brain or spinal tissue from a fish selected from the group consisting of parrotfish, sea bass, turbot, grouper, stripped jack, tiger puffer, berfin flounder, halibut, barramundi, and spotted wolffish.

24. The method according to claim 13, wherein said virus is infectious pancreatic necrosis virus (IPNV).

25. The method according to claim 24, wherein said fish tissue is pancreatic or intestinal tissue from a fish susceptible fish is one selected from the group trout, salmon, carp, perch, pike, and eel.
